(19) United States
(12) Patent Application Publication
(10) Pub. No.: US 2009/0291913 A1
(43) Pub. Date: Nov. 26, 2009

(54) FACTOR Xa INHIBITOR FORMULATION AND METHOD

(75) Inventors: Munir N. Nassar, Skillman, NJ (US); Uday S. Gogate, North Brusnwick, NJ (US); Timothy M. Malloy, Yardley, PA (US)

Correspondence Address: Fitzpatrick Cella (BMS), 1290 Avenue of the Americas, New York, NY, 10104-3800, US

(73) Assignee: Bristol-Myers Squibb Company, Princeton, NJ (US)

(21) Appl. No.: 12/535,016

(22) Filed: Aug. 4, 2009

Related U.S. Application Data
(63) Continuation of application No. 11/464,519, filed on Aug. 15, 2006.
(60) Provisional application No. 60/709,077, filed on Aug. 17, 2005.

(51) Int. Cl. A61K 31/724; A61P 7/02

(52) U.S. Cl. 514/58

(57) ABSTRACT

An injectable Factor Xa inhibitor formulation is provided which includes the Factor Xa inhibitor razaxaban or apixaban, a solubilizing agent which is a substituted β-cyclodextrin, preferably, sulfobutyl ether β-cyclodextrin (SBE-CD) or hydroxypropyl-β-cyclodextrin (HPB-CD), and water. A method for preventing or treating venous thrombosis, deep venous thrombosis and acute coronary syndrome employing the above formulation is also provided.

### CROSS-REFERENCE TO RELATED APPLICATIONS

The present application is a continuation of U.S. patent application Ser. No. 11/464,519, filed Aug. 15, 2006, which claims the priority benefit of U.S. Provisional Application No. 60/709,077, filed Aug. 17, 2005. Both prior applications are expressly incorporated fully herein by reference.

### FIELD OF THE INVENTION

The present invention relates to a Factor Xa inhibitor formulation which includes a Factor Xa inhibitor and a substituted-β-cyclodextrin solubilizing agent, a Factor Xa inhibitor inclusion complex with a substituted-β-cyclodextrin, an injectable formulation which contains a Factor Xa inhibitor and a substituted-β-cyclodextrin, and methods for inhibiting Factor Xa and preventing or treating venous thromboembolisms, deep vein thrombosis and acute coronary syndrome employing the above formulation.

### BACKGROUND OF THE INVENTION

U.S. Pat. No. 6,339,099 discloses the aminobenzisoxazole

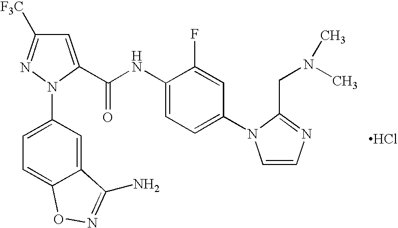

(hereinafter referred to as razaxaban) which inhibits the blood coagulation enzyme human Factor Xa and thus is useful in preventing or treating venous thromboembolism and deep vein thrombosis.

Razaxaban is a weak base with pH dependent solubility which shows decrease in solubility as the pH is increased. The neutral form or free base of razaxaban has extremely low solubility, which is estimated to be less than 1 μg/mL at room temperature at pH 6.8. Moreover, razaxaban in the form of its hydrochloride salt, at normal gastric pH condition, where the pH of the gastric medium is ˜1-2, has a solubility of ˜3 mg/mL.

The anticipated bolus human intravenous dose of razaxaban is about 50 mg. To achieve a practical injection volume, for example less than 20 mL, a solution with a high drug concentration, for example 2.5 mg/mL, is required. It has been found that solubility of razaxaban could not be increased to the needed level by adjusting pH to within a desirable pH range (pH 3-11). This pH range is desirable in order to minimize pain on injection of intravenous parenterals.

U.S. Patent Publication No. 2003/0191115 A1 (based on U.S. application Ser. No. 10/245,122 filed Sep. 17, 2002) discloses a series of Factor Xa inhibitors including 1-(4-methoxyphenyl)-7-oxo-6-[4-(2-oxo-1-piperidinyl)phenyl]-4,5,6,7-tetrahydro-1H-pyrazolo [3,4-c]pyridine-3-carboxamide (hereinafter referred to as apixaban), which has the structure:

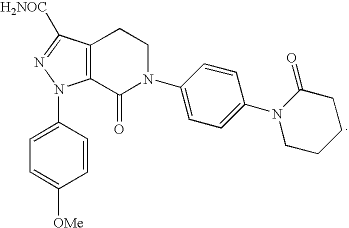

Apixaban is a weak base and is sparingly soluble (less than about 1 μg/mL at room temperature at pH 6.8).

Cyclodextrins are known for their use in increasing solubility of drugs. They function by forming inclusion complexes with hydrophobic molecules. Unfortunately, there are many drugs for which cyclodextrin complexation either is not possible or produces no apparent advantages as disclosed by J. Szejtli, Cyclodextrins in Drug Formulations: Part II, Pharmaceutical Technology, 24-38, August, 1991.

U.S. Pat. Nos. 5,134,127 and 5,376,645 each to Stella et al. disclose sulfoalkyl ether cyclodextrin derivatives and their use as solubilizing agents for water-insoluble drugs for oral, intranasal or parenteral administration including intravenous and intramuscular. Stella et al. disclose an inclusion complex of the water-insoluble drug and the sulfoalkyl ether cyclodextrin derivative and pharmaceutical compositions containing same. Examples of sulfoalkyl ether cyclodextrin derivatives disclosed include mono-sulfobutyl ether of β-cyclodextrin and monosulfopropyl ether of β-cyclodextrin. Examples of water-insoluble drugs are set out in column 7 starting at line 25.

U.S. Pat. No. 6,232,304 to Kim et al. discloses inclusion complexes of aryl-heterocyclic salts such as the tartrate salt of ziprasidone in a cyclodextrin such as β-cyclodextrin sulfobutyl ether (SBE-CD), and hydroxypropyl-β-cyclodextrin (HPBCD), and use of such inclusion complexes in oral and parenteral formulations.

U.S. Pat. No. 5,904,929 to Uekama et al. discloses trans-mucosal and transdermal pharmaceutical compositions containing a drug and a peracylated cyclodextrin as a solubilizing agent. Examples of drugs include anti-coagulants, namely, warfarin, and anti-stroke compounds such as lubetuzole, or its oxide, riluzole, aptiganel, eliprodil and remacemide.

U.S. Pat. No. 6,407,079 to Muller et al. discloses inclusion compounds formed of sparingly water-soluble and water unstable drugs and a β-cyclodextrin derivative. Muller et al. discloses employing a molar ratio of drug:β-cyclodextrin derivative from about 1:6 to 4:1, especially about 1:2 to a 1:1.

### BRIEF DESCRIPTION OF THE INVENTION

In accordance with the present invention, there is provided a formulation which includes a Factor Xa inhibitor such as razaxaban or apixaban, and a solubilizing agent which is a substituted-β-cyclodextrin. It has been found that the substituted beta-cyclodextrin increases solubility of the Factor Xa inhibitor sufficiently to allow formulation of an aqueous injectable containing 2.5 mg/mL or more of the Factor Xa inhibitor in a volume of less than 20 mL so as to deliver 50 mg or more Factor Xa inhibitor in a single bolus injection.

Surprisingly and unexpectedly, it has been found that the Factor Xa inhibitor such as razaxaban and apixaban and a substituted-β-cyclodextrin such as sulfobutyl ether-β-cyclodextrin may be formulated as an injectable which delivers the Factor Xa inhibitor with acceptable injection volumes to a muscular site.

The Factor Xa inhibitor for use herein are defined by the following genuses.

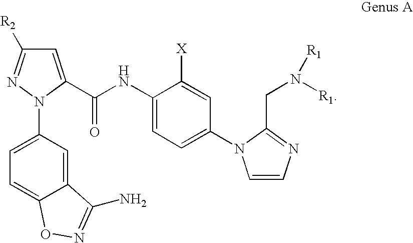

and pharmaceutically acceptable salts thereof,
wherein R₂ is alkyl or polyhaloalkyl, preferably CF₃;

R₁ is alkyl, preferably CH₃; and

X is halogen, preferably F.

Genus A set out above is covered by the genus of compounds disclosed in U.S. Pat. No. 6,339,099, which is incorporated herein by reference, and includes the Factor Xa inhibitors disclosed and/or generically covered in U.S. Pat. No. 6,339,099.

A preferred Factor Xa inhibitor for use herein within the Genus A is razaxaban which has the structure

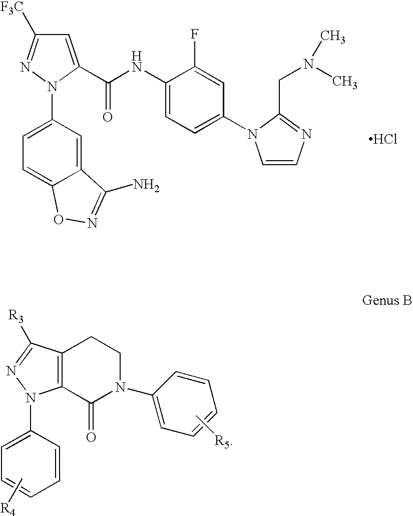

and pharmaceutically acceptable salts thereof, wherein R₃ is selected from

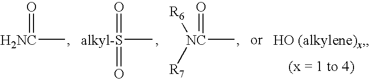

(where R₆ and R₇ are the same or different and are alkyl) preferably

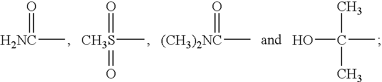

R₄ is selected from alkoxy and halogen, preferably methoxy; and

R₅ is

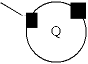

wherein Q is a 6 membered monocyclic ring wherein 0, 1 or 2 double bonds are present within the ring and the ring is substituted with 0, 1 or 2 R₅ₐ groups which at each occurrence is independently selected from H, ═0 or alkyl, and

Q₁ is C═O.

Preferred R₅ groups are

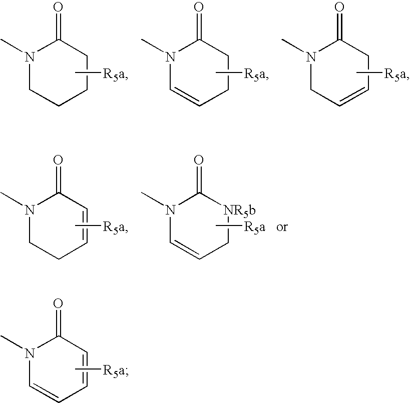

wherein R₅ₐ, at each occurrence, is independently selected from H, ═O, CH₃, CH₂CH₃, CH₂CH₂CH₃, CH(CH₃)₂, CH₂CH₂CH₂CH₃, CH₂CH(CH₃)₂, CH(CH₃)CH₂CH₃ and C(CH₃)₃; and

R_{5b} is H or alkyl, such as CH₃, CH₂CH₃, CH₂CH₂CH₃, CH(CH₃)₂, CH₂CH₂CH₂CH₃, CH₂CH(CH₃)₂, CH(CH₃)CH₂CH₃ and C(CH₃)₃.

R₅ is preferably

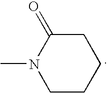

Genus B set out above is covered by the genus of compounds disclosed in U.S. Patent Publication No. 2003/0191115 A1, which is incorporated herein by reference, and includes the Factor Xa inhibitors disclosed in and/or generically covered by U.S. Patent Publication No. 2003/0191115 A1.

A preferred Factor Xa inhibitor for use herein within the Genus B is apixaban which has the structure

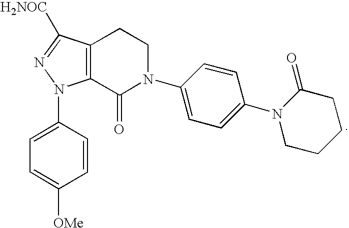

The compounds within the scope of Genuses A and B are collectively referred to herein as “the Factor Xa inhibitor(s)”.

In addition, in accordance with the present invention, a pharmaceutical formulation is provided which is formed of the Factor Xa inhibitor and a substituted-β-cyclodextrin, and a pharmaceutically acceptable carrier therefor.

In a preferred embodiment, the pharmaceutical formulation of the invention will be in the form of an aqueous parenteral or injectable formulation. However, the pharmaceutical formulation of the invention may be in other dosage forms such as lyophilized injectable, oral (for example tablets, capsules, elixirs and the like), transdermal or transmucosal forms or inhalation forms.

The injectable formulation of the invention will preferably be a clear colorless to light yellow solution, essentially free of particulate matter by visual inspection.

Further, in accordance with the present invention, a method is provided for administering injectable Factor Xa inhibitor without causing unacceptable irritation at the site of injection wherein the above described injectable formulation is administered, preferably intramuscularly, to a patient in need of treatment.

Still further in accordance with the present invention, a method is provided for inhibiting the blood coagulation enzyme human Factor Xa and for preventing or treating venous thromboembolism, deep vein thrombosis and acute coronary syndrome, which includes the step of administering to a patient in need of treatment the above described formulation, preferably in injectable form, without causing undue irritation at the site of injection, whether it be at a muscular site or other site.

The desired Factor Xa inhibitor concentration of an injectable formulation in accordance with the present invention is a result of constraints on the bolus infusion volume of 20 mL (providing a maximum dose of 50 mg). The pH of the injectable formulation of the invention is an important consideration in determining maximum desired solubility of Factor Xa inhibitor and should be from about 3 to about 11, depending upon the particular Factor Xa inhibitor employed to minimize pain on injection.

Taking all of the above factors into consideration, in accordance with the present invention, it has been found that substituted-β-cyclodextrins, such as sulfobutyl ether β-cyclodextrin (SBE-CD) and hydroxypropyl-β-cyclodextrin (HPB-CD), are preferred solubilizing agents for the Factor Xa inhibitor.

The Factor Xa inhibitor razaxaban has the same solubility in the substituted-β-cyclodextrins at pH 4.5 and at higher pH's such as up to 11. In fact, it has been found that by lowering pH of the razaxaban-substituted-β-cyclodextrin solution to between about 3 and about 4, increase in solubility of razaxaban is achieved and the desired injectable drug concentration and volume may be obtained without causing undue irritation or pain at the site of injection.

The desired pH of the injectable formulation of the invention containing compounds of Genus A such as razaxaban is obtained by use of acid buffers and base. The lower pH limit will be about 3. pHs below 3 are undesirable due to physiological constraints such as irritation at the site of injection. The upper pH limit will be about 11 to provide a safety margin with respect to drug solubility. However, a pH within the range from about 3 to about 5 is preferred to achieve desired injectable drug concentration and volume.

The desired pH of the injectable formulation of the invention containing compounds of Genus B such as apixaban is obtained by use of buffers to adjust pH of the aqueous injection within the range from about 6 to about 8, preferably about 7.

### DETAILED DESCRIPTION OF THE INVENTION

Factor Xa inhibitors of the Genuses A and B set out above such as razaxaban and apixaban have poor water solubility and thus are difficult to formulate as aqueous injectables. In accordance with the present invention, it has been found that the water-solubility of the Factor Xa inhibitors may be sufficiently increased to allow it to be formulated as an aqueous injectable by employing the Factor Xa inhibitor with a substituted-β-cyclodextrin solubilizing agent. This is indeed surprising and unexpected since a host of water-miscible co-solvent systems and water-immiscible co-solvent systems have been found to be unacceptable as carriers for injectable Factor Xa inhibitors such as razaxaban, because they do not increase solubility of the Factor Xa inhibitor sufficiently to provide for a drug concentration of at least 2.5 mg/mL at an acceptable injection volume. On the other hand, the aqueous injectable formulation of the invention delivers the Factor Xa inhibitor such as razaxaban or apixaban in at least a 2.5 mg/mL concentration in 20 mL or less volume to provide an acceptable dose such as 50 mg or more for razaxaban and 5 mg or more for apixaban in a single bolus injection.

As will be seen hereinafter, the Factor Xa inhibitor formulation of the invention in the form of an aqueous injectable will include a buffer to adjust pH to desired levels.

The substituted-β-cyclodextrin suitable for use herein refers to sulfobutyl ether β-cyclodextrin (SBE-CD) and hydroxypropyl-β-cyclodextrin (HPB-CD), with SBE-CD being preferred.

The term “bolus” as used herein refers to a single injection containing a full dose of drug, which is administered over a relatively short period of time, such as one minute or less.

The term “undue irritation” or “unacceptable irritation” at the site of injection or at the muscular site refers to moderate to severe irritation which is unacceptable to the patient and thereby impacts unfavorably on patient compliance.

The term “reduced irritation” at the site of injection or at the muscular site refers to generally minimal to mild irritation which is acceptable to the patient and does not impact unfavorably on patient compliance.

The term “acute coronary syndrome” as used herein refers to a person experiencing chest pain which may be due to an attack of unstable angina or a heart attack.

Unless otherwise indicated, the term “lower alkyl”, “alkyl” or “alk” as employed herein alone or as part of another group includes both straight and branched chain hydrocarbons, containing 1 to 10 carbons, preferably 1 to 8 carbons, in the normal chain, such as methyl, ethyl, propyl, isopropyl, butyl, t-butyl, isobutyl, pentyl, hexyl, isohexyl, heptyl, 4,4-dimethylpentyl, octyl, 2,2,4-trimethylpentyl, nonyl, decyl, undecyl, dodecyl, the various branched chain isomers thereof, and the like as well as such groups including 1 to 4 substituents such as halo, for example F, Br, Cl or I or CF₃, alkoxy, aryl, aryloxy, aryl(aryl) or diaryl, arylalkyloxy, alkenyl, cycloalkyl, cycloalkylalkyl, cycloalkylalkyloxy, hydroxy, hydroxyalkyl, acyl, arylalkoxycarbonyl, aryloxyalkyl, aryloxyaryl, alkylamido, alkanoylamino, arylcarbonylamino, nitro, cyano, thiol, haloalkyl, trihaloalkyl and/or alkylthio.

(alkylene)ₓ includes alkylene of 1 to 4 carbons in the normal chain, which may optionally include 1, 2, or 3 substituents which include alkyl, alkenyl, halogen, cyano, hydroxy, alkoxy, amino, thioalkyl, keto, C₃-C₆ cycloalkyl, alkylcarbonylamino or alkylcarbonyloxy; the alkyl substituent may be an alkyl moiety of 1 to 4 carbons which may be attached to one carbon in the (CH₂)ₓ.

Examples of (alkylene)ₓ include

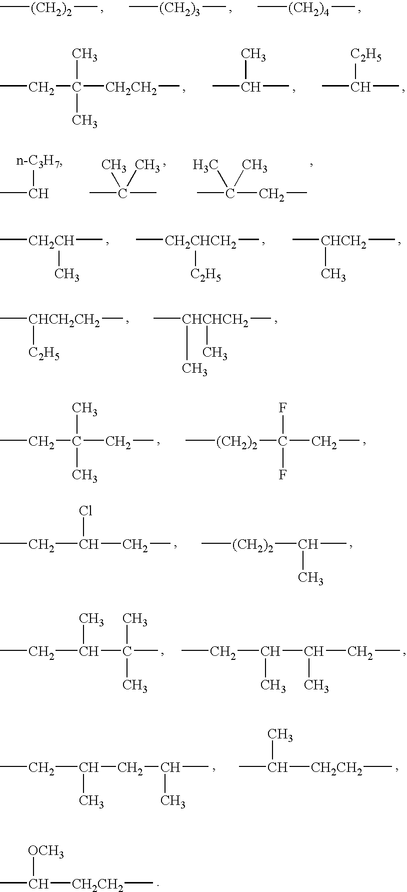

The term “halogen” or “halo” as used herein alone or as part of another group refers to chlorine, bromine, fluorine, and iodine as well as CF₃, with chlorine or fluorine being preferred.

The term “polyhaloalkyl” as used herein refers to an “alkyl” group as defined above which includes from 2 to 9, preferably from 2 to 5, halo substituents, such as F or Cl, preferably F, such as CF₃CH₂, CF₃ or CF₃CF₂CH₂.

It is believed that the Factor Xa inhibitor will form a complex with the substituted-β-cyclodextrin which complex may be dissolved in water to form an injectable formulation. However, physical mixtures of the Factor Xa inhibitor and the substituted-β-cyclodextrin and aqueous solutions formed directly (without redissolving a solid formulation of the Factor Xa inhibitor and the substituted-β-cyclodextrin) are within the scope of the present invention as well.

The complex or the physical mixture may also be compressed into a tablet or may be filled into capsules.

The Factor Xa inhibitor formulations of the invention may be formed directly as aqueous solutions or as dry physical mixtures of the Factor Xa inhibitor and the substituted-β-cyclodextrin or dry inclusion complexes thereof which upon addition of water may be reconstituted to form an aqueous injectable formulation. Alternatively, the aqueous injectable formulation may be freeze dried and later reconstituted with water. Thus, the Factor Xa inhibitor formulation in accordance with the invention, may be pre-formed, formed in situ or formed in vivo (in the gastrointestinal tract or the buccal cavity). All of the above are contemplated by the present invention.

Where the Factor Xa inhibitor employed in the formulation of the invention in the form of an aqueous injectable is a weak base, such as razaxaban, the formulation will include an acid buffer to adjust pH of the aqueous injection within the range from about 3 to about 9, preferably from about 3 to about 5. Examples of acid buffers suitable for use herein include acids such as hydrochloric acid, sulfuric acid, phosphoric acid, hydrobromic acid and the like, and organic acids such as oxalic acid, maleic acid, fumaric acid, lactic acid, malic acid, tartaric acid, citric acid, benzoic acid, acetic acid, methanesulfonic acid, toluenesulfonic acid, benzenesulfonic acid, ethanesulfonic acid and the like. Acid salts of the above acids may be employed as well. Preferred acids are tartaric acid, citric acid, phosphoric acid and hydrochloric acid. Most preferred is citric acid.

The injectable formulation of the invention containing the Factor Xa inhibitor razaxaban will have a pH within the range from about 3 to about 9, preferably from about 3 to about 5, and more preferably from about 3 to about 3.4, and most preferably about 3.2. In formulating the injectable, if necessary, the pH may be adjusted with a base such as an alkali metal citrate such as sodium citrate, or potassium citrate, an alkali metal hydroxide such as NaOH, KOH, or LiOH, preferably NaOH, or an alkaline earth metal hydroxide, such as Mg(OH)₂ or Ca(OH)₂, with sodium citrate being preferred.

Where the Factor Xa inhibitor is in the form of a free base such as the Factor Xa inhibitor apixaban, the formulation will include a buffer to adjust pH of the aqueous injection within the range from about 6 to about 8, preferably about 7.

Examples of such buffers suitable for use herein include phosphate buffer (that is dihydrogen phosphate and sodium hydroxide, or a mixture of dibasic sodium phosphate and monobasic sodium phosphate), and tris buffer (that is hydroxymethyl aminoethane), which buffers will adjust pH as indicated above to provide maximum stability.

In preparing the aqueous injectable formulation of the invention, the substituted-β-cyclodextrin will be employed in a molar ratio to the Factor Xa inhibitor such as razaxaban or apixaban within the range from about 5:1 to 400:1, preferably from about 10:1 to about 100:1. Each type of cyclodextrin employed requires a different ratio to provide acceptable drug concentration.

In preferred embodiments of the aqueous injectable of the invention, the substituted-β-cyclodextrin will be SBE-CD which will be employed in a molar ratio to Factor Xa inhibitor such as razaxaban or apixaban within the range from about 5:1 to about 400:1, preferably from about 10:1 to about 80:1, more preferably 12:1 (based on a razaxaban concentration of 2.5 mg/mL and 12% w/v SBE-CD (120 mg/mL)). The cyclodextrin may be present in an amount greater than that needed to complex the Factor Xa inhibitor since the additional cyclodextrin could aid in dissolution of the drug.

In another preferred embodiment of the invention, SBE-CD will be employed in a molar ratio to apixaban within the range from about 50:1 to about 100: 1, preferably about 70:1 to about 90: 1, more preferably about 75:1 (based on a drug concentration of 1 mg/mL drug and 35% w/v SBE-CD (350 mg/mL)).

In still another preferred embodiment of the invention, hydroxypropyl-β-cyclodextrin (HPB-CD) will be employed in a molar ratio to apixaban within the range from about 30:1 to about 100: 1, preferably from about 40:1 to about 70: 1, more preferably about 45:1 (based on a drug concentration of 2.5 mg/mL and 35% w/v HPB-CD (350 mg/mL)).

The Factor Xa inhibitor will be present in the aqueous injectable formulation in an amount within the range from about 0.1 to about 2% by weight, preferably from about 0.2 to about 1% by weight based on the total injectable formulation.

In preferred embodiments, the Factor Xa inhibitor will be present in the aqueous injectable formulation to provide from about 1 to about 20 mg/mL of formulation, preferably from about 2 to about 10 mg/mL of formulation, and more preferably at least about 2.5 mg/mL up to about 8 mg/mL of formulation.

In more preferred embodiments, the formulations of the invention will provide 2.5 mg razaxaban/mL or 2.5 mg apixaban/mL, 5 mg/mL and 7.5 mg/mL. Fill volumes will preferably be 10 mL and 20 mL for razaxaban, and 2 mL, 4 mL and 10 mL for apixaban.

A preferred injectable formulation is as follows:

(1) razaxaban—in an amount to provide from about 2.5 to about 8 mg/mL of solution.

(2) SBE-CD—in an amount from about 50 to about 200 mg/mL of solution.

(3) acid buffer (preferably citric acid)—in an amount from about 0.5 to about 5 mg/mL of solution to adjust pH from about 3 to about 5.

(4) base to adjust pH, preferably an alkali metal citrate, preferably sodium citrate, in an amount to adjust pH from about 3 to 5.

(5) water qs to 1 mL.

The razaxaban injectable formulation of the invention may be prepared as follows: Citric acid or other acid as described herein and base such as sodium citrate or other base as described herein are dissolved in water for injection. The substituted-β-cyclodextrin (preferably SBE-CD) is dissolved in the buffered aqueous solution. Razaxaban is then dissolved in the solution. Additional water for injection is added to obtain the desired batch volume.

The resulting solution is aseptically filtered, for example, through a 0.22μ membrane filter and filled into vials. The vials are stoppered and sealed and may be terminally sterilized.

The aqueous injectable formulation of the invention will provide an amount of razaxaban of at least 2 mg razaxaban/mL, preferably at least 2.5 mg razaxaban/mL, when the amount of razaxaban provided by the complex is measured at a cyclodextrin concentration of 5-20% w/v in water.

Another preferred injectable formulation is as follows:

(1) apixaban—in an amount to provide from about 2.5 to about 8 mg/mL of solution.

(2) HPB-CD—in an amount from about 50 to about 500 mg/mL of solution.

(3) Phosphate buffer (dihydrogen phosphate and sodium hydroxide or dibasic sodium phosphate and monobasic sodium phosphate)—in an amount from about 0.5 to about 5 mg/mL of solution to adjust pH from about 6 to about 8.

(4) water qs to 1 mL.

The Factor Xa inhibitor apixaban injectable formulation of the invention may be prepared as follows: Phosphate buffer or tris buffer is dissolved in water for injection. The substituted-β-cyclodextrin (preferably HPB-CD or SBE-CD) is dissolved in the buffered aqueous solution. Apixaban is then dissolved in the solution. Additional water for injection is added to obtain the desired batch volume.

The resulting solution is aseptically filtered, for example, through a 0.22μ membrane filter and filled into vials. The vials are stoppered and sealed and may be terminally sterilized.

The aqueous injectable formulation of the invention will provide at least 2 mg apixaban/mL, preferably at least 2.5 mg apixaban/mL, when the amount of apixaban provided by the complex is measured at a cyclodextrin concentration of 35% w/v in water.

The formulations of the invention are used to inhibit Factor Xa and prevent or treat diseases associated with Factor Xa including venous thrombosis, deep vein thrombosis and acute coronary syndrome in human patients. The preferred dosage employed for the injectable formulations of the invention will be a 2 to 20 ml injection containing 2.5 mg razaxaban/mL or 2.5 mg apixaban/mL or a dose of 25 to 50 mg razaxaban given once daily or 2.5 to 10 mg apixaban given once daily. The injectable formulation is preferably administered intramuscularly although subcutaneous and intravenous injections are effective as well.

The following Examples represent preferred embodiments of the invention.

### EXAMPLE 1

A clear colorless razaxaban injectable solution (2.67 mg razaxaban/mL, 10.5 mL/vial) essentially free of particulate matter by visual inspection having the following composition was prepared as follows.

TABLE 1
Quantitative Composition of Razaxaban Injection,
25 mg/vial (2.5 mg/mL) as the Free Base

| | Rationale | | | | |
|---|---|---|---|---|---|
| Ingredient | for Use | Amount Per mL | | Amount Per Vial | |
| Razaxaban | Active | 2.67 ^{b} | | 28.06 | mg ^{a, b} |
| | Ingredient | | | | |
| Captisol ™ | Solubilizer | 120 | mg | 1260 | mg |
| (SBE-CD) | | | | | |
| Citric Acid | Stabilizer | 1.831 | mg | 19.23 | mg |
| USP/EP | (buffer) | | | | |
| (monohydrate) | | | | | |
| Sodium Citrate, | Stabilizer | 0.379 | mg | 3.98 | mg |
| USP/EP | (buffer) | | | | |
| (Dihydrate) | | | | | |

| | | | |
|---|---|---|---|
| Water for | Solvent | q.s. to 1.0 mL | q.s. to 10.5 mL ^{a} |
| Injection, | | | |
| USP/EP | | | |

^{a} Target fill volume is 10.5 mL. This volume includes a 0.5 mL overfill for Vial-Needle Syringe (VNS) holdup.
^{b} Assuming 100% purity. The 28.06 mg of razaxaban (hydrochloride salt, MW = 564.92) is equivalent to 26.25 mg of the Free Base (MW = 528.46). The 2.67 mg of razaxaban (hydrochloride salt) is equivalent to 2.50 mg of the Free Base.

A stainless steel batching vessel was charged with an amount of water for injection USP/EP (WFI) equal to about 85% of the final batch volume.

With continuous mixing, citric acid monohydrate granular USP and sodium citrate USP/EP were added to the batching vessel and stirred until a completed solution was obtained.

With continuous mixing, sulfobutyl ether β-cyclodextrin (Captisol™) (about 1.26 kg) were added to the batching vessel and stirred until a complete solution was obtained.

Razaxaban (about 28 g) was added to the batching vessel and stirring was continued until the razaxaban was dissolved and a complete solution was obtained.

Additional water for injection USP was added to the above solution to adjust to the final batch size of 10.5 L with stirring.

The above bulk solution was aseptically filtered through a 0.22 μM porosity sterilizing filter into a sterile receiving container. 10.5 mL amounts of the above solution were aseptically filled into sterile 15 cc flint type 1 tubing glass vials which were then aseptically stoppered with sterilized stoppers to seal the vials.

The razaxaban injectable solution prepared above had a pH ranging from about 3.1 to about 3.3 at 20°-25° C. with a target pH of 3.2 at 20°-25° C., a bulk solution density of 1.047 g/mL of 23° C. and a solution potency ranging from about 2.42 mg/mL to about 2.58 mg/mL as the free base with a target potency of 2.5 mg/mL as the free base.

### EXAMPLE 2

A clear colorless to light yellow apixaban injectable solution (2.5 mg drug/mL, 2 mL/vial) essentially free of particulate matter by visual inspection having the following composition was prepared using hydroxypropyl β-cyclodextrin (HPB-CD) as follows.

TABLE 2
Quantitative Composition of Apixaban,
5 mg/vial (2.5 mg/mL) as the Free Base

| | Rationale | | | | |
|---|---|---|---|---|---|
| Ingredient | for Use | Amount Per mL | | Amount Per Vial | |
| Apixaban | Active | 2.5 | mg | 5.5 | mg ^{a} |
| | Ingredient | | | | |
| HPB-CD | Solubilizer | 350 | mg | 770 | mg |
| Sodium Phosphate | Stabilizer | 0.83 | | 1.826 | |
| Monobasic | (buffer) | | | | |
| (monohydrate) | | | | | |
| Sodium Phosphate | Stabilizer | 0.57 | mg | 1.254 | mg |
| Dibasic (anhydrous) | (buffer) | | | | |

| | | | |
|---|---|---|---|
| Water for Injection, | Solvent | q.s. to 1.0 mL | q.s. to 2.2 mL ^{a} |
| USP/EP | | | |

^{a} Target fill volume is 2.2 mL. This volume includes a 0.2 mL overfill for Vial-Needle Syringe (VNS) holdup.

### Apixaban Injectable Solution

A 10 mM phosphate buffer pH ˜7 was prepared as follows:

0.8001 Grams of sodium phosphate monobasic was dissolved in 400 mL water and volume was q.s to 500 mL (pH 4.57).

0.7099 Grams of sodium phosphate dibasic was dissolved in 400 mL water and volume was q.s to 500 mL (pH 9.2). 400 mL of the 10 mM dibasic sodium phosphate was placed in a 1-L beaker and 400 mL of the monobasic sodium phosphate solution was added. Final pH was 7.01.

17.5 Grams of HPB-CD was dissolved in 30 mL of the 10 mM phosphate buffer, pH 7. 0.125 Grams of apixaban was added to the solution and the solution was mixed until solids mixed until dissolved. A sufficient quantity of the 10 mM phosphate buffer solution was added to bring the final volume to 50 mL.

The above bulk solution was aseptically filtered through a 0.22 μm porosity sterilizing filter into a sterile receiving container. 2.2 mL amounts of the above solution were aseptically filled into sterile 5 cc glass vials which were then aseptically stoppered with sterilized stoppers to seal the vials.

The apixaban injectable solution prepared above had a pH about 7 at 20°-25° C. which was the target pH, a bulk solution density of 1.102 g/mL at about 23° C. and a solution potency ranging from about 2.25 mg/mL to about 2.75 mg/mL as the free base with a target potency of 2.5 mg/mL as the free base.

### EXAMPLE 3

A clear colorless to light yellow apixaban injectable solution (1 mg apixaban/mL, 5.2 mL/vial) essentially free of particulate matter by visual inspection having the following composition was prepared using SBE-CD as follows.

TABLE 3
Quantitative Composition of Apixaban Injection,
5 mg/vial (1 mg/mL) as the Free Base

| | Rationale | | | | |
|---|---|---|---|---|---|
| Ingredient | for Use | Amount Per mL | | Amount Per Vial | |
| Apixaban | Active | 1 | mg | 5.2 | mg ^{a} |
| | Ingredient | | | | |
| Captisol ™ (SBE-CD) | Solubilizer | 350 | mg | 1820 | mg |
| Sodium Phosphate | Stabilizer | 0.83 | mg | 4.32 | mg |
| Monobasic | (buffer) | | | | |
| (monohydrate) | | | | | |
| Sodium Phosphate | Stabilizer | 0.57 | mg | 2.96 | mg |
| Dibasic (anhydrous) | (buffer) | | | | |

| | | | |
|---|---|---|---|
| Water for Injection, | Solvent | q.s. to 1.0 mL | q.s. to 5.2 mL ^{a} |
| USP/EP | | | |

^{a} Target fill volume is 5.2 mL. This volume includes a 0.2 mL overfill for Vial-Needle Syringe (VNS) holdup.

17.5 Grams of SBE-CD was dissolved in 30 mL of 10 mM phosphate buffer pH 7 (prepared as in Example 2). 0.05 Grams of apixaban was added to the solution and the solution mixed until solids were dissolved. A sufficient quantity of the 10 mM phosphate buffer pH 7 was added to bring the final volume to 50 mL.

The above bulk solution was aseptically filtered through a 0.22 μm porosity sterilizing filter into a sterile receiving container. 5.2 mL amounts of the above solution were aseptically filled into sterile 10 cc glass vials which were then aseptically stoppered with sterilized stoppers to seal the vials.

The apixaban injectable solution prepared above had a pH about 7 at 20°-25° C. which was the target pH, a bulk solution density of 1.102 g/mL of 23° C. and a solution potency ranging from about 0.90 mg/mL to about 1.10 mg/mL as the free base with a target potency of 1 mg/mL as the free base.

What is claimed is:

1. A pharmaceutical formulation comprising apixaban and HPB-CD.

2. The formulation according to claim 1, which is an aqueous injectable formulation having a pH within a range from about 6 to about 8.

3. The formulation according to claim 2, including a phosphate buffer or a tris buffer.

4. The formulation according to claim 1, wherein apixaban is present in an amount to provide a dosage from about 2 to 8 mg drug/mL.

5. The formulation according to claim 1, wherein a weight ratio of HPB-CD to apixaban is from about 20:1 to about 40:1.

6. An inclusion complex of apixaban in HPB-CD.

7. An aqueous injectable formulation comprising apixaban, HPB-CD, buffer, and water, the formulation having a pH within a range from about 6 to about 8.

8. The formulation according to claim 7, comprising apixaban in an amount from about 2 to about 8 mg/mL of the formulation; HPB-CD in an amount from about 100 to about 500 mg/mL of the formulation; sodium phosphate monobasic monohydrate in an amount from about 0.5 to about 2 mg/mL of the formulation; sodium phosphate dibasic is present in amount from about 0.4 to about 1.5 mg/mL of the formulation, to adjust a pH within a range from about 6 to about 8; and water qs to 2 mL.

9. The formulation according to claim 7, which is an inclusion complex, wherein the inclusion complex provides an amount of apixaban of at least 2 mg apixaban/mL when the amount of apixaban provided by the complex is measured at an HPB-CD concentration of 35 w/v in water.

10. The formulation according to claim 7, comprising apixaban in an amount of about 2.5 mg/mL of the formulation; HPB-CD in an amount of about 350 mg/mL of the formulation; sodium phosphate monobasic monohydrate in amount of about 0.83 mg/mL of the formulation; sodium phosphate dibasic in amount of about 0.57 mg/mL of the formulation; and water qs to about 1.0 mL of the formulation.

11. The formulation according to claim 7, comprising apixaban in an amount of about 1.0 mg/mL of the formulation; SBE-CD in an amount of about 350 mg/mL of the formulation; sodium phosphate monobasic monohydrate in amount of about 0.83 mg/mL of the formulation; sodium phosphate dibasic in amount of about 0.57 mg/mL of the formulation; and water qs to about 1.0 mL.

12. A method for administering an injectable Factor Xa inhibitor to a patient in need of treatment without causing unacceptable irritation at a site of injection, the method comprising administering to a patient in need of treatment the formulation of claim 1.

13. A method of preventing or treating venous thromboembolism, deep vein thrombosis or acute coronary syndrome, which comprises administering to a patient in need of treatment the formulation of claim 1.
